(19) United States
(12) Patent Application Publication
(10) Pub. No.: US 2025/0000818 A1
(43) Pub. Date: Jan. 2, 2025

(54) NEW SYNERGISTIC COMBINATIONS BASED ON FENM AND AN ACETYLCHOLINESTERASE INHIBITOR

(71) Applicants: REST Therapeutics, Paris (FR); Institut National de la Sante et de la recherche Medicale, Paris (FR); UNIVERSITE DE MONTPELLIER, MONTPELLIER (FR); ECOLE PRATIQUE DES HAUTES ETUDES, Paris (FR)

(72) Inventors: Gilles RUBINSTENN, PARIS (FR); Tangui MAURICE, SAINT-GELY-DU-FESC (FR); Aline FREYSSIN, ADISSAN (FR)

(21) Appl. No.: 18/708,355

(22) PCT Filed: Nov. 8, 2022
(86) PCT No.: PCT/EP2022/081184

§ 371 Date: May 8, 2024

(30) Foreign Application Priority Data
Nov. 8, 2021 (FR) FR2111841

(51) Int. Cl. A61K 31/13; A61K 31/445; A61K 45/06

(52) U.S. Cl. CPC A61K 31/13; A61K 31/445; A61K 45/06

(57) ABSTRACT

The invention relates to a composition comprising a synergistic combination of fluoroethylnormemantine (FENM) and at least one acetylcholinesterase inhibitor. More particularly, the invention relates to said composition for use thereof as a drug, even more particularly for use thereof in the treatment of a pathology selected from among tauopathies, synucleinopathies, amyloidopathies, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis.

The invention relates to the field of neurodegenerative diseases.

More particularly, the invention relates to compositions comprising a synergistic combination of 3 (2-Fluoroethyl)tricyclo[3.3.1.13,7]decan-1-amine (Fluoroethylnormemantine, FENM) with at least one acetylcholinesterase inhibitor.

The invention also relates to these combinations or compositions comprising them for use thereof in the treatment of neurodegenerative pathologies and, more particularly, in the prevention and/or treatment of the cognitive disorders of these pathologies.

### PRIOR ART

The World Health Organization (WHO) estimates that by 2050 the number of people over the age of 60 will reach two billion. This unprecedented aging of the world's population suggests that chronic age-related diseases will put high pressure on healthcare systems. Dementia is one of these. Thus, the WHO estimates that the total number of people living with dementia is expected to exceed 150 million by 2050.

Dementia is characterized by a deterioration in the cognitive functions, in particular the memory and the reasoning, which impacts the patient's behavior and his/her ability to perform daily tasks. Dementia is a syndrome that involves pathologies with a wide variety of etiologies that affect different areas of the brain and/or other regions of the central nervous system, including in particular the neurodegeneration and the death of neural cells. Directly related to aging, mitochondrial dysfunction and oxidative stress play a crucial role in the pathogenesis of neurodegenerative diseases. These pathologies and syndromes are also often related to abnormal accumulation of certain proteins and/or accumulation of mutated and/or abnormally folded proteins as observed in AB amyloidosis, tauopathies, synucleinopathies, superoxide dismutase-1 (SOD1) aggregation, polyglutamine, TDP-43 protein.

Alzheimer's disease is the most common cause of dementia and is thought to cause 60-70% of cases (WHO source).

Today, several molecules are allowed in the symptomatic treatment of Alzheimer's disease. These include anticholinesterases such as donepezil, rivastigmine, or galantamine which benefit from marketing authorizations, as monotherapy, in the mild, moderate, or moderately severe stage of the disease. Memantine, a noncompetitive voltage-dependent antagonist of NMDA receptors, is allowed in the moderate and severe stages of the disease and is not allowed in the mild stage of the disease. These compounds are not recommended in the early stage of the disease due to a lack of clinical efficacy. In addition, their effects are symptomatic and limited and have only been demonstrated in the short term (average 6 months) in nearly two thirds of patients included in the clinical studies (source, Haute Autorité de Santé, France). A combination treatment of donepezil and memantine, under the brand name Acrescent®, had its marketing authorization (MA) refused by the European Medicines Agency (EMA) due to the low benefits of this combination (20 mg memantine/10 mg donepezil) with regards to the compounds used alone (EMA, Oct. 18, 2012). The only interest would be to take a single pill instead of two, in patients with cognitive impairment; this treatment has nevertheless been approved in the US by the FDA. Furthermore, the alleged efficacy of anticholinesterases in the early stage of the disease is based on a single study performed specifically on patients at this stage, in which a modest benefit was observed in the only patients who tolerated doses of 10 mg/day of donepezil. The health authorities in France consider that this study does not allow concluding on the interest of starting this treatment at the mild stage of the disease (Source Haute Autorité de Santé, France, 2012).

Acetylcholinesterase inhibitors are also associated with a risk of serious adverse effects or those that may alter quality of life and/or require discontinuation of treatment. These may include digestive disorders (diarrhea, vomiting), cardiovascular disorders (bradycardia, syncope), or even neuropsychiatric disorders (vertigo, mental confusion) that are counterproductive in the case of treatment of dementia.

The risk of adverse effects which increases with the duration of treatment, in addition to the lack of benefit beyond six months, strongly prevails the possibility of use thereof in the short term. In practice, these treatments are applied to patients with already impaired cognitive abilities, in the moderate or even advanced stage of the disease.

The application WO 2014/191424 describes the FENM marked at ¹⁸F for the marking of NMDA receptors and their visualization by positron emission tomography to study the distribution of these receptors and their reaction to drug treatments. The application WO 2019/115833 describes FENM in the treatment of anxiety-related disorders and depression.

The application WO 2013/064579 describes a combination of a connexin blocking agent (such as meclofenamic acid) with an acetylcholine esterase inhibitor (such as donepezil) for use thereof in the treatment of cognitive impairment.

Aducanumab is an anti-amyloid monoclonal antibody that is used to decrease the amyloid load in the brain. Aducanumab received an MA issued by the FDA in 2021 for the treatment of Alzheimer's disease in the early or prodromal stage with proven presence of amyloid deposits. Given the non-reproduced results concerning a lesser cognitive decline in treated patients, and taking into account the absence of a therapeutic solution, the MA was conditioned on a re-evaluation of the product to confirm its clinical interest (<www.fda.gov>). Approximately 35% of treated patients presented cerebral microhemorrhages and cerebral edema (source France Alzheimer, <www.francealzheimer.org>). The cost of the treatment, which requires monthly intravenous administrations, is estimated at 56,000 dollars per year.

The diagnosis of dementia or Alzheimer's disease at an early stage leads to a significant need for treatment by the patient and their family to stop or slow the progression of the disease as early as possible. Nowadays, this need is still unmet. There is also an important need for more effective treatments for both the treated symptoms and the efficacy duration of the treatment in patients at a later stage of the disease.

### Technical Problem

The invention aims to remedy the drawbacks of the prior art. In particular, the invention aims to propose a new therapy for cognitive impairments in neurodegenerative diseases and in particular Alzheimer's disease (AD). This new therapy is based on the combined action of fluoroethylnormemantine (FENM) and at least one acetylcholinesterase inhibitor. The synergistic action identified between these molecules allows, on the one hand, obtaining particularly effective effects in the preservation of cognitive abilities and/or reducing the doses used for these compounds. This decrease allows reducing the risk of adverse effects or even increasing the possible duration of the treatment while achieving greater improvement at particularly low doses. This improved efficacy allows considering the use of this treatment, even at an early stage of the disease.

### BRIEF DESCRIPTION OF THE INVENTION

The Applicants have quite surprisingly discovered that the combination of the effects of FENM with those of acetylcholinesterase inhibitors has a synergistic effect in the treatment of cognitive symptoms in an intoxicated mouse model by intracerebral administration of amyloid peptide oligomers β₂₅₋₃₅ (Aβ₂₅₋₃₅). This synergistic action is observed in cognitive tests of short-term working memory, but also of long-term contextual memory. Such synergistic effects allow considering treatments that are more effective than those currently applied and broadening the population of patients eligible for these treatments compared to current practices.

Consequently, one object of the present invention relates to a composition comprising a synergistic combination of 3-(2-fluoroethyl) adamantan-1-amine (FENM) or any one of its pharmaceutically-acceptable salts and at least one acetylcholinesterase inhibitor or any one of its pharmaceutically-acceptable salts.

In a particular embodiment, said at least one acetylcholine esterase inhibitor is selected from among donepezil, galantamine, rivastigmine, tacrine, or any one of their pharmaceutically-acceptable salts. Indeed, these molecules benefit from an MA or have already been tested in humans and their pharmacokinetic and pharmacodynamic properties are already known, which is a particular advantage.

According to other features of the composition according to the invention:
the compounds are formulated together or separately.
the FENM and/or said at least one acetylcholinesterase inhibitor is mixed with a pharmaceutically-acceptable excipient.

The synergistic interaction between FENM and the acetylcholinesterase inhibitors discovered by the Inventors allows considering, compared to monotherapies, a better therapeutic management of patients, with a gain in treatment efficacy and/or a reduction in the doses administered and therefore a reduction in the possible side effects associated with the active compounds of the composition of the invention, namely FENM and the at least one acetylcholinesterase inhibitor. Nevertheless, at these low doses, the therapeutic benefit is maintained and even improved.

Thus, in particular and independent embodiments, in the composition according to the invention:
the FENM dose is compatible with a FENM dosage less than or equal to 20 mg/day,
said at least one acetylcholinesterase inhibitor is donepezil which is present at a dose compatible with a dosage less than or equal to 10 mg/day,
said at least one acetylcholinesterase inhibitor is rivastigmine which is present at a dose compatible with a dosage less than or equal to 3 mg/day, and/or
said at least one acetylcholinesterase inhibitor is galantamine which is present at a dose compatible with a dosage less than or equal to 16 mg/day.

In another one of its particular embodiments, in the composition according to the invention, the molar ratio FENM/at least one acetylcholine esterase inhibitor is less than or equal to 4, less than or equal to 3, preferably less than or equal to 2, preferably less than or equal to 1.

A particular object of the present invention relates to the composition as described hereinabove, in any one of its embodiments, for use as a drug.

More particularly, and as demonstrated in the experimental part, the composition comprising a synergistic combination of FENM and at least one acetylcholinesterase inhibitor is particularly effective for counteracting the cognitive impairments in a neurodegenerative pathology model. Thus, another object of the invention relates to said composition in any one of its above-described embodiments for use thereof in the treatment of a pathology selected from among tauopathies, synucleinopathies, amyloidopathies, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis.

In the composition of the invention, for the purposes of any one of the previously-described uses, FENM and the at least one acetylcholinesterase inhibitor are administered separately, concomitantly or sequentially. For example, this may be operated by using specific formulations of the FENM on the one hand and the at least one acetylcholinesterase inhibitor on the other hand resulting in differentiated infusion kinetics for each of the compounds included in a unit composition; alternatively according to another example, the at least FENM on the one hand and the at least one acetylcholinesterase inhibitor of the combination on the other hand are formulated and administered in individualized galenic forms.

Another object of the invention is FENM, or a pharmaceutically-acceptable salt thereof, in synergistic combination with at least one acetylcholinesterase inhibitor selected from among donepezil, galantamine, rivastigmine, tacrine or any one of their pharmaceutically-acceptable salts, for use in the treatment of a pathology selected from among tauopathies, synucleinopathies, amyloidopathies, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis.

### FIGURES

FIG. 1 (A) symptomatic effect of FENM, donepezil (DPZ) or the FENM-DPZ combination on memory impairment induced by the oligomerized Aβ₂₅₋₃₅ peptide in the Y-labyrinth test; Data±standard error. ANOVA: F_{(14,200)}=2.511, p=0.0026, n=30-31 per group for non-intoxicated controls treated with the vehicle and 11-20 for the treated groups. **°p<°0.01, ***p<0.001 vs. treated with the vehicle and non-intoxicated; #p<0.05, ##p<0.01, ###°p°<0.001 vs. Intoxicated with the Aβ₂₅₋₃₅oligomers and treated with the vehicle; Dunnett's test. (B) Protection scale showing the level of protection (PP) conferred by donepezil (DPZ), FENM or the FENM-Donepezil mix (MIX). The calculated combination index is less than 1. The 100% correspond to the performance level of mice not intoxicated by the Aβ₂₅₋₃₅ oligomers and treated with the vehicle. 0% protection corresponds to the performance level of intoxicated mice treated in the vehicle. S: synergistic effect; V: vehicle.

FIG. 2 (A) symptomatic effect of donepezil FENM (DPZ) or the FENM-DPZ combination on memory impairment induced by intoxication with oligomerized Aβ25-35 peptide in the passive avoidance test. The results are non-parametric data and are presented as a median and 25%-75% deviations. Training takes place on day 9, 48 hours after the last treatment. Retention was tested on day 10, 24 hours after training. Kruskal-Wallis ANOVA: H°=°54.27, p<0.0001, n=34-36 per group for non-intoxicated control animals treated with the vehicle and 11-20 for the groups intoxicated with the Aβ₂₅₋₃₅ oligomers and treated with the compounds. *p<0.05, **p<0.01, *** p<0.001 vs. non-intoxicated and vehicle-treated group; #p<0.05, ##p<0.01, ## #p<0.001 vs. group intoxicated with Aβ₂₅₋₃₅ and treated with the vehicle; Dunn test. (B) Protection scale showing the level of protection (PP) conferred by donepezil (DPZ), FENM or the FENM-donepezil MIX (MIX). The calculated combination index is less than 1. The 100% correspond to the performance level of mice not intoxicated by the Aβ₂₅₋₃₅ oligomers and treated with the vehicle. 0% protection corresponds to the performance level of intoxicated mice treated in the vehicle. S: synergistic effect; V: vehicle.

### DESCRIPTION OF THE INVENTION

### Definitions

In the context of the present invention, mention of a specific drug or compound includes not only the drug or compound specifically named, but also any salt, hydrate, derivative, isomer, racemate, enantiomerically-pure composition, conjugate or corresponding pharmaceutically-acceptable prodrug of the active molecule of the drug or said compound. Preferably, mention of a compound includes the specifically named compound, as well as any salt, hydrate, isomer, racemate, isomer, enantiomerically pure, pharmaceutically-acceptable composition of said compound. More preferably, the designation of a compound is intended to designate the compound as specifically designated in itself, as well as any pharmaceutically-acceptable salt thereof. Nevertheless, unless stated otherwise, the mention in unit of mass or in unit of mass per day of the amount of a compound of the composition or the combination according to the invention, means the designated compound itself.

By “pharmaceutically-acceptable salts”, it should be understood, within the meaning of the invention, a pharmaceutically-acceptable and relatively non-toxic inorganic or organic acid addition salt of a compound of the present invention. Pharmaceutical salt formation involves coupling an acidic, basic, or zwitterionic drug molecule with a counterion to create a saline version of the drug. A wide variety of chemical species can be used in the neutralization reaction. Hence, the pharmaceutically-acceptable salts of the invention include those obtained by reacting the concerned compound, when it functions as a base, with an inorganic or organic acid to form a salt, for example, salts of acetic acid, nitric acid, tartaric acid, hydrochloric acid, hydrobromic acid, sulfuric acid, phosphoric acid, methane sulfonic acid, camphor sulfonic acid, oxalic acid, maleic acid, succinic acid or citric acid. The pharmaceutically-acceptable salts of the invention also comprise those in which, when the concerned compound functions as an acid, said compound is reacted with a suitable base to form, for example, salts of sodium, potassium, calcium, magnesium, ammonium or choline. Although most salts of a given active ingredient are bioequivalent, some may have, inter alia, increased solubility or bioavailability properties. Salt selection is now a common standard operation in the drug development process as taught by Stahl and Wermuth in their handbook (Stahl and Wermuth).

Examples of compounds usable to implement a combination of FENM with acetylcholinesterase inhibitors according to the invention are listed in Table 1 hereinbelow.

TABLE 1

| Compound | CAS Reference Number^{†} |
|---|---|
| FENM | 1639210-26-6 |
| FENM HBr | NA |
| FNEM HCl | NA |
| donepezil | 120014-06-4; 120011-70-3 |
| galantamine | 357-70-0; 1953-04-4; 5072-47-9 |
| rivastigmine | 123441-03-2; 129101-54-8 |
| tacrine | 321-64-2; 1684-40-8; 7149-50-0 |

^{†}Chemical Abstracts Service database registration number.
NA: not applicable.

A preferred donepezil salt is donepezil hydrochloride.

A preferred galantamine salt is galantamine hydrobromide.

A preferred rivastigmine salt is rivastigmine tartrate.

A preferred FENM salt is FENM hydrochloride.

A particularly preferred FENM salt is FENM hydrobromide.

The term “combination” within the meaning of the present invention means a treatment in which at least FENM and at least one acetylcholinesterase inhibitor is co-administered to a subject to cause a biological effect. In a combination therapy according to the invention, these at least two compounds can be administered together or separately, at the same time or sequentially. In particular, FENM and said at least one acetylcholinesterase inhibitor may be administered following different routes and/or administration protocols. Consequently, although they could be formulated together, the compounds as elements of a combination within the meaning of the invention may also be formulated separately. For example, FENM may be administered orally and said at least one acetylcholinesterase inhibitor of the combination according to the invention may be injected into said subject, for example, intravenously, subcutaneously or transdermally. In another embodiment, for example, FENM may be administered orally and said at least one acetylcholinesterase inhibitor may also be administered orally to said subject concurrently or in a time-shifted manner. Preferably, the sequence of administration of the active ingredients of the combination (FENM and said at least one acetylcholinesterase inhibitor) is such that said active ingredients or their active metabolite(s) exert their biological effects at the same time, such that the subject benefits from the maximum effect of said combination. Thus, in a particularly preferable manner, FENM and said at least one acetylcholinesterase inhibitor are administered so as to reach their maximum concentration in plasma or cerebrospinal fluid, preferably cerebrospinal fluid, at the same time.

The term synergy applied to combinations according to the invention means combinations for which the observed or known pro-mnesic or anti-amnesic effects for each of the active ingredients of said combination are added up or multiplied when used so that their physiological effects interact. In particular, this synergistic effect allows obtaining pro-mnesic or anti-amnesic effects at doses at which the active ingredients applied in monotherapies would have no or limited effect, which could allow reducing the side effects and/or avoiding or shortening the duration of dose-escalation protocols. Furthermore, this synergy between the effects of the compounds of the combination according to the invention also allows potentiating these pro-mnesic or anti-amnesic effects or improving complex memory types as shown in the experimental part. Methods for analyzing the interaction between two molecules and determining the synergistic effect of a combination of compounds are well known to a person skilled in the art. A non-limiting example is the analysis of the isobolograms and the determination of the combination index according to the principles set forth by Fraser (1872) and as implemented, for example, by Martin et al. (2020). Thus, a presence of synergy can be detected by applying the mathematical method illustrated in the experimental part. Synergy can be demonstrated in vivo in animal models of a pathology, for example via tests to assess the cognitive performances or morphological impairments associated with the pathology. The synergy can be demonstrated in vitro, for example, in the context of in vitro cell tests, in models recognized by the scientific community such as for example cytotoxicity tests, by measuring various parameters relating to cell viability, and in the case of neural cells, growth of neurites, formation of synapses, etc. As regards dementia and/or AD, illustrations of these tests are given, for example, by Chumakov et al. (2015).

By “subject”, it should herein be understood any member of the animal kingdom, preferably mammals and even more preferably humans. A subject in need of the combination treatments of the invention is defined as a subject suffering, suspected of suffering, or considered at risk of suffering from a neurodegenerative pathology leading to dementia and associated cognitive impairments. For example, these conditions are tauopathies, synucleinopathies, amyloidopathies, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis. The compositions, combinations and methods of the present invention are particularly suitable for the treatment of cognitive disorders associated with Alzheimer's disease in its early, moderate or advanced stage. Thus, in a particular embodiment, said subject suffers, is suspected of suffering, or is considered at risk of suffering from Alzheimer's disease in its early, moderate, or advanced stage. In another particular embodiment, said subject suffers, is suspected of suffering, or is considered at risk of suffering from cognitive disorders associated with the early, moderate, or advanced stages of Alzheimer's disease, preferably cognitive disorders associated with the early stage of Alzheimer's disease.

As used herein, the term “treatment” includes the therapy, the prevention, the prophylaxis, the delay or the reduction of the symptoms caused by or the causes of the diseases or disorders hereinabove. In particular the term treatment encompasses, monitoring the progression of the disease and of the associated symptoms. In particular, the term treatment encompasses protection against the effects of toxicity caused by amyloid B, or a reduction or delay of these effects in treated subjects. In particular, the term treatment refers to an improvement, cessation or delay in the evolution of the cognitive symptoms of the aforementioned diseases.

### Combination of FENM and at Least One Acetylcholinesterase Inhibitor

As shown by the experimental data presented hereinbelow, a synergistic interaction of the pro-mnesic or anti-amnesic effects of FENM and acetylcholinesterase inhibitors has been discovered, resulting in an improvement in cognitive symptoms in an experimental animal model of Aβ₂₅₋₃₅-induced toxicity. The protective effect of this combination against cognitive decline caused by amyloid peptide intoxication Aβ₂₅₋₃₅ is observed for both short-term working memory and long-term memory.

Thus, in a first aspect, the invention relates to a synergistic combination of FENM or any one of its pharmaceutically-acceptable salts and at least one acetylcholinesterase inhibitor or any one of its pharmaceutically-acceptable salts.

In a particular embodiment, said at least one acetylcholinesterase inhibitor is selected from among donepezil, galantamine, rivastigmine or tacrine. Donepezil, galantamine and rivastigmine are particularly preferred because they have been approved for more than twenty years, so their side effects and pharmacological properties are well known. Due to its hepatotoxicity, tacrine is less preferred. Nevertheless, the synergistic effect highlighted herein which concerns protection against cognitive symptoms may allow considering the use of low doses at which this hepatotoxicity is not observed. The synergistic combination of FENM with donepezil is particularly preferred.

As mentioned, the synergistic effect observed for the combinations of the invention allows considering using, in humans, the minimum doses or doses lower than these minimum doses at which the acetylcholinesterase inhibitors are used in humans.

Thus, in a particular embodiment, in the synergistic combination according to the invention, the at least one acetylcholinesterase inhibitor is donepezil and is administered at a dose less than or equal to 10 mg per day, less than or equal to 5 mg per day, less than or equal to 2.5 mg per day, but still sufficient to observe a beneficial effect of the combination on the subject's cognitive abilities.

In another particular embodiment, in the synergistic combination according to the invention, the at least one acetylcholinesterase inhibitor is galantamine and is administered at a dose less than or equal to 16 mg per day, less than or equal to 8 mg per day, less than or equal to 4 mg per day or less than 2 mg per day, but still sufficient to observe a beneficial effect of the combination on the subject's cognitive abilities.

In another particular embodiment, in the synergistic combination according to the invention the at least one acetylcholinesterase inhibitor is rivastigmine and is administered at a dose less than or equal to 3 mg per day, less than or equal to 1.5 mg per day, or less than or equal to 1 mg per day, but still sufficient to observe a beneficial effect of the combination on the subject's cognitive abilities.

In a particular embodiment, FENM, in the synergistic combination according to the invention, is used at a dose less than or equal to 20 mg per day, less than or equal to 10 mg per day, or less than or equal to 5 mg per day, but still sufficient to observe a beneficial effect of the combination on the subject's cognitive abilities.

The cognitive abilities of human subjects and their evolution can be measured by tests well known to a person skilled in the art. Tests commonly used for the cognitive assessment of human subjects are, for example, the Mini-Mental State Examination (MMSE or Folstein's test), Modified Mini-Mental State Examination (or 3 MS scale), Abbreviated Mental Test Score (AMTS or Abbreviated Mental Test), Dementia questionnaire for persons with Mental Retardation (or DMR questionnaire), Cognitive Abilities Screening Instrument (CASI), Trail-making test, Clock drawing test, Alzheimer's disease assessment scale-Cognition (ADAS-Cog), General Practitioner Assessment of Cognition (GPCOG), Montreal Cognitive Assessment (MoCA), or the Rowland Universal Dementia Assessment Scale (RUDAS), or the Alzheimer's Disease Cooperative Study-Activities of Daily Living (ADCS-ADL).

More particularly, MMSE allows people with major neurocognitive impairment (dementia) to be screened without being tied to a particular pathology. MMSE is also used to monitor people's cognitive condition and measure the decline in the cognitive functions in people with neurocognitive impairment. This test evaluates orientation, recording, attention and calculation, mnesia retention, language and building practice. The CERAD (the acronym of the Consortium to Establish a Registry for Alzheimer's Disease) established a dementia severity scale associated with scores obtained in the MMSE. A score between 19 and 24 is associated with mild dementia, between 10 and 18, a moderate dementia, and a score below 10 corresponds to a severe dementia, the maximum score being 30. A 2-point change in the score is generally considered clinically relevant.

ADAS-Cog is a cognitive subscale of the Alzheimer's disease scale and therefore concerns only cognition-related aspects of dementia. Thus, it can be used to assess (i.e. score) and monitor the progress of any type of dementia. ADAS-Cog assesses orientation, memory, executive function, visuospatial ability, language, or practice, with a score range of 0 to 70, a higher score indicating greater deficiency. ADAS-Cog is considered more sensitive than MMSE. It is one of the most commonly used tests for the clinical assessment of compound candidates for Marketing Authorization in anti-dementia treatments and also to measure the evolution of cognitive impairments.

Thus, a combination with a beneficial effect of the combination on the subject's cognitive abilities will show a slowdown, or a stabilization, of the subject's deterioration in cognitive abilities with regards to the usual course established in untreated subjects at the same stage of the disease for a given period of time. In other words, for example, in the case of ADAS-Cog, a beneficial effect on the subject's cognitive abilities will correspond to a decrease, a stabilization, or an increase of less intensity in the ADAS-Cog score compared to the usual worsening of the score observed in untreated subjects at an equivalent stage and with an equivalent age. Although the worsening of the score depends on the stage of the pathology and the age of the subject, an annual increase by 5.82 points in the ADAS-Cog score is generally observed in subjects with untreated Alzheimer's disease (Zhang et al., 2020). As regards MMSE, for example, a beneficial effect on the subject's cognitive abilities will correspond to a lower increase, a stabilization, or a decrease in intensity of the MMSE score compared to the usual worsening of the MMSE score observed in untreated subjects. An annual decrease by 2.28-point in the MMSE score is generally observed in subjects with untreated Alzheimer's disease (Rossetti et al. 2010).

Surprisingly, synergy between FENM and at least one acetylcholinesterase inhibitor is observed for a very low FENM/acetylcholinesterase inhibitor molar ratio. Indeed, the molar ratio tested for memantine and donepezil in the state of the art is greater than 4, if we consider the combinations based on 20 mg memantine and 10 mg donepezil), this is in particular the ratio contained in NAMZARIC® approved by the FDA, based on donepezil hydrochloride and memantine hydrochloride. It should be noted that the Tmax values in humans for donepezil and memantine when administered orally are comparable: between 3 and 8 hours for memantine (Maekawa et al., 2019) and 4.1±1.5 hours for donepezil (Rogers & Friedoff, 1998). The experimental data show that the synergistic effect discovered for the combination of the invention is obtained at a much lower molar ratio, which may even be reversed, reflecting the specificity of the synergistic combinations based on FENM.

Thus, in a particular embodiment of the synergistic combination of FENM and at least one acetylcholinesterase inhibitor according to the invention, the FENM/acetylcholinesterase inhibitor molar ratio is less than or equal to 4, less than or equal to 3, less than or equal to 2, preferably less than or equal to 1, less than or equal to 0.8, or less than or equal to 0.5. Said FENM/acetylcholinesterase inhibitor molar ratio being greater than or equal to 0.1. In a particularly preferable manner, said FENM/acetylcholinesterase inhibitor molar ratio is less than 1 and greater than or equal to 0.1. In another particular embodiment, said acetylcholinesterase inhibitor is donepezil and the FENM/donepezil molar ratio is less than or equal to 4, is less than or equal to 3, less than or equal to 2, preferably less than or equal to 1, less than or equal to 0.8, or less than or equal to 0.5. Said FENM/donepezil molar ratio being greater than or equal to 0.1. In a particularly preferable manner, said FENM/donepezil molar ratio is less than 1 greater than or equal to 0.1.

### Composition According to the Invention Comprising a Synergistic Combination of FENM and at Least One Acetylcholinesterase Inhibitor.

In another aspect, the invention relates to a composition comprising a synergistic combination as described hereinabove.

In this composition, the FENM or any one of its pharmaceutically-acceptable salts and the at least one acetylcholinesterase inhibitor or any one of its pharmaceutically-acceptable salts are formulated together or separately.

In a particular mode, FENM and the at least one acetylcholinesterase inhibitor are the only active ingredients of said composition. In other words, FENM and the at least one acetylcholinesterase inhibitor are the only compounds within the composition having a therapeutic or preventive activity.

Preferably, the composition is administered to the subject in the form of a pharmaceutical preparation, for example, without limitation, orally, locally (cutaneously, orally, sublingually) or parenterally (subcutaneously, intramuscularly or intravenously). Oral administration is particularly preferred.

The amounts of the active ingredients within this composition, i.e. at least FENM and at least one acetylcholinesterase inhibitor, are compatible with the doses as determined hereinabove for the synergistic combinations of the invention. In other words, when said composition is in the form of a unit dosage (in the form of a pill, capsule, powder, emulsion, solution), said dosage comprises amounts of FENM or of the at least one acetylcholinesterase inhibitor that are a multiple or a divider of the doses determined for the synergistic combinations of the invention, thereby allowing obtaining in one or more doses the appropriate dosage as defined above. In a particular embodiment, the composition according to the invention comprises 10 mg donepezil, 7.5 mg donepezil, 5 mg donepezil, or 2.5 mg donepezil. In another particular embodiment, the composition according to the invention comprises 16 mg galantamine, 8 mg galantamine, 4 mg galantamine, or 2 mg galantamine. In another particular embodiment, the composition according to the invention comprises 3 mg rivastigmine, 1.5 mg rivastigmine, or 1 mg rivastigmine.

In a particular embodiment, the composition according to the invention comprises 20 mg FENM, 10 mg FENM, 7.5 mg FENM, 5 mg FENM, or 2.5 mg FENM.

In another embodiment, the composition according to the invention comprises 20 mg FENM and 10 mg donepezil, 7.5 mg donepezil, 5 mg donepezil, or 2.5 mg donepezil. In another embodiment, the composition according to the invention comprises 10 mg FENM and 10 mg donepezil, 7.5 mg donepezil, 5 mg donepezil, or 2.5 mg donepezil. In another embodiment, the composition according to the invention comprises 7.5 mg FENM and 10 mg donepezil, 7.5 mg donepezil, 5 mg donepezil, or 2.5 mg donepezil. In another embodiment, the composition according to the invention comprises 5 mg FENM and 10 mg donepezil, 7.5 mg donepezil, 5 mg donepezil, or 2.5 mg donepezil. In another embodiment, the composition according to the invention comprises 2.5 mg FENM and 10 mg donepezil, 7.5 mg donepezil, 5 mg donepezil, or 2.5 mg donepezil.

In another embodiment, in the composition according to the invention, the FENM/acetylcholinesterase inhibitor molar ratio is less than or equal to 4, less than or equal to 3, less than or equal to 2, preferably less than or equal to 1, less than or equal to 0.8, or less than or equal to 0.5. Said ratio is greater than or equal to 0.1. In a particularly preferable manner, said FENM/acetylcholinesterase inhibitor molar ratio is less than 1 and greater than or equal to 0.1. In another particular embodiment, said acetylcholinesterase inhibitor is donepezil and the FENM/donepezil molar ratio is less than or equal to 4, is less than or equal to 3, is less than or equal to 2, preferably less than or equal to 1, less than or equal to 0.8, or less than or equal to 0.5, said ratio being greater than or equal to 0.1. In a particularly preferable manner, said FENM/donepezil molar ratio is less than 1 and greater than or equal to 0.1.

The dose may be administered in multiple doses distributed throughout the day, the number of doses throughout the day allowing obtaining the desired daily dose. Thus, in a particular embodiment, the considered doses may be administered in one to four daily dose(s), for example 1 time, for example 2 times, for example 3 times, or 4 times.

In a preferred embodiment, in said composition, the combination of FENM or of the at least one acetylcholinesterase inhibitor is conditioned so as to provide the dose corresponding to one dose without requiring any manipulation such as a volume measurement, weighing or division of a tablet, which is particularly advantageous in subjects with cognitive impairments since this avoids any particular calculation or manipulation.

For example, FENM or at least one acetylcholinesterase inhibitor is formulated separately in the form of powder, microgranules, granules or pills that are distinct and then combined into a capsule to facilitate intake.

The pharmaceutical compositions may be formulated according to conventional pharmaceutical practice (cf., for example, Remington: The Science and Practice of Pharmacy (23rd ed.), ed. Adeboye Adejare, 2020) and the PK/PD characteristics of the active ingredients. Conventionally, in the compositions according to the invention, the FENM and/or said at least one acetylcholinesterase inhibitor is mixed with a pharmaceutically-acceptable excipient.

In one embodiment, said composition is in the form of a tablet or pill. Said tablet or pill may be splitable into 1, 2, 3, or 4 piece(s) so as to be able to provide the subject with the dose necessary for an intake using 1, 2 or 3 piece(s) of said tablet. This is particularly interesting, for example, in the case where the treatment requires a period of dose increase, to reach the target daily dose, the pieces may correspond to the increase steps, and the whole tablet or pill to the target dose of the treatment.

In one embodiment, in said composition, FENM and the at least one acetylcholinesterase inhibitor are mixed within the same tablet or pill with the same excipient(s). In a particular embodiment, the FENM and the at least one acetylcholinesterase inhibitor are present in compartments different from said tablet or pill with excipients that are specific thereto, and which depend on their physicochemical properties or their pharmacokinetic properties.

In another embodiment, particularly interesting in the case of molecules with different PK/PD profiles, FENM and the at least one acetylcholinesterase inhibitor are present separately within this tablet. Thus, in a particular embodiment, FENM and the at least one acetylcholinesterase inhibitor are present in compartments different from said tablet or pill, for example one compound being located outside and the other compound being located inside said tablet or pill, which enables a separate and time-shifted administration of these compounds.

In an embodiment of the composition according to the invention, FENM and/or the at least one acetylcholinesterase inhibitor may be formulated to be released substantially immediately after administration, at any time, or at a predetermined period of time after administration i.e. formulated so as to be released in a controlled manner into the body. Controlled-release formulations include (i) formulations which create a substantially constant concentration of the compound or its active derivative in the body over an extended period of time; (ii) formulations which, after a predetermined latency time, create a substantially constant concentration of the compound or its active derivative in the body over an extended period of time; (iii) formulations which maintain the action of the active compound or its derivative for a predetermined period of time by maintaining a relatively constant and effective level of said compound or its active derivative in the body, further enabling a concurrent minimization of undesirable side effects associated with the fluctuations in the plasma level of said compound or its active derivative; (iv) formulations which localize the action of the compound or its active derivative, for example, near or in the diseased tissue or organ, or in a specific body compartment; and (v) formulations which target the action of said compound or its active derivative using carriers or chemical derivatives to deliver the drug to a particular target cell type.

The administration of compounds in the form of a controlled-release formulation is particularly preferred in cases where the compound has (i) a narrow therapeutic index (i.e. the difference between the plasma concentration leading to harmful effects, side effects or toxic reactions and the plasma concentration leading to a therapeutic effect is low; in general, the therapeutic index, TI, is defined as the ratio of the median lethal dose (DL50) to the median effective dose (DE50)); (ii) a narrow window of absorption in the gastrointestinal tract; or (iii) a very short biological half-life so that frequent administrations are necessary to maintain the plasma level at an effective therapeutic level. Different strategies may be pursued in order to obtain a release with a release rate of the compound or its active derivative adapted to the metabolism of the considered drug. Controlled release may be achieved by an appropriate selection of various formulation parameters and ingredients, including, for example, various types of controlled release compositions and coatings known to a person skilled in the art. Thus, the compound is formulated with suitable excipients into a pharmaceutical composition which, upon administration, releases said compound in a controlled manner (unit or multiple compositions of pills or capsules, oily solutions, suspensions, emulsions, microcapsules, microspheres, nanoparticles, patches and liposomes).

In an even more particular embodiment, specific technical means such as reservoirs, pumps or transdermal patches (in other words, a self-adhesive patch that percutaneously dispenses a substance) could contribute to the controlled release of at least FENM and/or the at least one acetylcholinesterase inhibitor.

In another embodiment, said composition is formulated in a liquid form. It may be packaged in the form of a unit dose in containers such as ampoules, or in a container such as a bottle or a vial associated with a device enabling sampling and, optionally, the delivery of the desired volume to obtain the appropriate dose.

### Therapeutic Uses of the Synergistic Combination or of the Composition According to the Invention.

The new synergistic effect discovered by the Inventors for the combination of FENM at least one acetylcholinesterase inhibitor as described in the experimental part forms a new therapeutic solution.

Thus, according to another aspect, the invention relates in particular to the composition or synergistic combination as described before in all embodiments thereof, for use thereof as a drug. In a preferred embodiment, the invention relates to the synergistic combination of FENM and donepezil or a composition comprising it, as described hereinabove in all embodiments thereof, for use thereof as a drug.

According to a further aspect, the invention relates particularly to the composition or to the synergistic combination, as described before, for use thereof in the treatment of a pathology selected from tauopathies, synucleinopathies, amyloidopathies, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis. In a particular embodiment, said treatment relates to the treatment of Alzheimer's disease in its early, moderate or advanced stage, preferably Alzheimer's disease in its early stage. In a particular embodiment, said treatment relates to the treatment of cognitive disorders associated with any one of the pathologies selected from among tauopathy, synucleinopathy, amyloidopathy, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis.

A particular embodiment relates to the composition or the synergistic combination as previously described for use thereof in the treatment of cognitive disorders associated with the early, moderate or advanced stage of Alzheimer's disease, preferably the early stage. Indeed, the experimental data show a particularly important synergistic effect in the treatment of long-term contextual memory impairments, which has a particular advantage in the case of patients in the early stages of the disease.

Another particular embodiment relates to the composition or synergistic combination as described before for use thereof for the alterations:
of the short-term memory,
of the mid-term memory,
of the spatial memory, or
of the recognition and/or learning abilities,
   associated with any one of the pathologies selected from among tauopathy, synucleinopathy, amyloidopathy, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis. In a particular embodiment, said impairments of the short-term memory, the medium-term memory, the spatial memory, or the recognition and/or learning abilities are associated with the early, moderate or advanced stage of Alzheimer's disease, preferably the early stage.

According to another aspect, the invention relates to a method for treating a pathology selected from among tauopathies, synucleinopathies, amyloidopathies, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis comprising administering to a subject in need, a synergistic combination of FENM or any one of its pharmaceutically-acceptable salts with at least one acetylcholinesterase inhibitor or any one of its pharmaceutically-acceptable salts.

In said method, said synergistic combination is as described before, in all embodiments. In one embodiment, said method may comprise administering a composition according to the invention as described hereinabove.

In a particular embodiment, said method comprises the separate, concomitant or sequential administration of FENM and the at least one acetylcholinesterase inhibitor. In this embodiment, FENM and the at least one acetylcholinesterase inhibitor may be administered separately to the subject via identical routes, such as for example via the oral, parenteral or transdermal route. In an alternative embodiment, FENM and the at least one acetylcholinesterase inhibitor may be administered to the subject via different routes. For example, one by the oral route and the other by the transdermal or parenteral route. A particular example is the transdermal form of rivastigmine marketed under the name Exelon® or donepezil (in clinical trials in the United States under the name Adlarity®).

### Examples

### Abbreviations

Aβ₂₅₋₃₅: fragment of 11 amino acids of the Nt-GSNKGAIIGLM-Ct sequence (SEQ ID NO 1) of the APP peptide.
FENM: 3 (2-Fluoroethyl)tricyclo[3.3.1.13,7]decan-1-amine bromohydrate (also fluoroethylnormemantine bromohydrate).
DPZ: donepezil hydrochloride.
YMT: spontaneous alternation test in the Y-labyrinth; “Y-maze test”.
PAT: passive avoidance test; “Passive Avoidance Test” or “step-through passive avoidance test”.
ICV: intracerebroventricular.
IP: intraperitoneal.
CI: combination index.
iChE: acetylcholinesterase inhibitor.
PP: percentage of protection.
CNS: Central Nervous System.
AD: Alzheimer's disease.

### 1. Equipment and Methods

The animal experiments are conducted in accordance with the provisions of the European Union Directive No. 2010/63 and have been duly authorized by the National Ethics Advisory Committee (CCNE) of the French Republic and the ARRIVE directives (Kilkenny et al., 2010).

### 1.1. Animals

In vivo experiments were conducted on male Swiss OF-1 mice (January, St Berthevin, France) aged 7 to 9 weeks with a mass of 32±2 g. The animals are housed in groups of 8 to 10 individuals in plastic cages with free access to food and drink, in a controlled environment (12:00 day/night cycle, the light is switched on at 7:00 a.m. and the behavioral experiments take place between 9:00 a.m. and 5:00 p.m.), under controlled atmosphere and sound environment.

### 1.2. Test Compounds and Peptides

### DPZ and FENM Stock Solutions.

The DPZ was obtained from Eisai Co. Ltd (Tokyo, Japan). FENM was obtained from M2i Life Sciences (Saint-Cloud, France). The stock solutions of the compounds were obtained by dissolution in NaCl buffer (0.9%, vehicle) at a concentration of 2 mg/mL which corresponds to a dose of 5 mg/Kg. These stock solutions are stored at 4° C. for 2 weeks maximum.

### Stock Solution of Amyloid Peptide [25-35]; Formation of Oligomers.

The amyloid peptide [25-35], denoted Aβ₂₅₋₃₅ (Eurogentec, Angers, France), was solubilized in sterile distilled water at a concentration of 3 mg/mL, the stock solution thus formed is aliquoted and stored at −20° C. until use.

The Aβ₂₅₋₃₅ oligomers are formed as described by Maurice et al. (1996), when incubated at 37° C. for 4 days before injection into animals. The vehicle solution or the control peptide are subjected to the same treatment before administration. It has already been demonstrated that the injection of vehicle solution (distilled water) results in the same absence of effect as the injection of control peptide Sc Aβ (control peptide), which comprises the same amino acids as Aβ₂₅₋₃₅ in a random order and which does not oligomerize.

### Administration to Animals

The compounds are IP administered in a volume of 100 μL per 20 g of body mass. The administration of the FENM/DPZ combination is performed in a volume of 100 μL per 20 g of body mass. The doses of compounds mentioned in the experimental part correspond to the doses of salts of the used compounds and not to the mass equivalent of the compound itself. The administered doses expressed in moles are listed in Table 2. For each compound, the administered doses are 0.01, 0.03, 0.1, 0.3 and 1 mg/kg. In the case of the combination, the co-administered doses of donepezil and FENM were 0.03 and 0.01 mg/kg, respectively.

The corresponding doses expressed in mMol/Kg are listed in Table 2 hereinbelow.

TABLE 2

| | Dose in mg/kg in the | |
|---|---|---|
| Active ingredient | experiments | Dose in mMol/Kg |
| fluoroethylnormemantine | 0.01 | 0.04 |
| | 0.03 | 0.11 |
| | 0.1 | 0.36 |
| | 0.3 | 1.08 |
| | 1 | 3.60 |
| donepezil | 0.03 | 0.07 |
| | 0.1 | 0.24 |
| | 0.3 | 0.72 |
| | 1 | 2.40 |
| | 3 | 7.21 |

The Aβ₂₅₋₃₅ oligomer solution and the vehicle (sterile distilled water) are administered to mice by ICV injection, as described in Maurice et al. (1996).

### 1.3. Cognitive/Behavioral Tests

The ICV injection model of Aβ₂₅₋₃₅ oligomers is a well-known model in the state of the art like. This model is considered to be a relevant screening model for neuroprotective activity of compounds, and more particularly a relevant first-line model of Alzheimer's disease. Aβ₂₅₋₃₅ oligomers are known to be cytotoxic to neural cells in mice and to induce spatial and working memory disorders. This deficiency is accompanied by the generation of mitochondrial stress, oxidative stress and apoptosis of cells, in particular in the hippocampus, and inflammation of the central nervous system.

The ability of the compounds and their combination to decrease the cognitive symptoms of neurodegeneration induced by the Aβ₂₅₋₃₅ oligomers is tested. For this purpose, the administration of the test compounds takes place on the same day as the injection of the Aβ₂₅₋₃₅ oligomers and continues on a daily basis until day 7 after the injection of the Aβ₂₅₋₃₅ oligomers; the mice are then subjected to YMT tests, or PAT on day 8 after the injection of the Aβ₂₅₋₃₅ oligomers.

### Passive Avoidance Test (PAT)

This test measures the long-term non-space (contextual) memory. The device used for this test is a box with two compartments (15×20×15 cm high), one illuminated with white PVC walls and the other in the dark with black PVC walls and a mesh floor. A guillotine door separates the compartments. A 60 W lamp is positioned 40 cm above the box and illuminates the white compartment. Electrical shocks (0.3 mA for 3 s) may be applied to the grid floor via a generator (Lafayette Instruments, Lafayette, USA). The test consists of a learning session and a test session. The guillotine door is closed during the training session. Each mouse is placed in the white compartment. After 5 seconds, the door is raised. When the mouse enters the dark compartment so that its four legs are in contact with the mesh, the door closes and electrical discharges are applied for 3 seconds. The time taken by the mouse to enter the dark compartment (STL-Tg) and the level of shock sensitivity (0=no sign; 1=surges; surge and vocalizes) are recorded. The test session is performed 24 hours after the learning session. Each mouse is placed back in the illuminated white compartment. After 5 seconds, the door is raised, the latency time (STL-R), i.e. the time it takes the mouse to go into the dark compartment, is measured. The maximum time is 300 seconds. Mice with STL-Tg and STL-R<10 s and low sensitivity level are not considered for the test. The attrition rate is usually 5%. A mouse with impaired memory capabilities will have a lower STL-R than a mouse with normal capabilities. The results are presented by their median value with the interquartiles (25%-75%).

### Y-Labyrinth Spontaneous Alternation Test (YMT)

The spontaneous alternation test is used to study the (very short-term) spatial working memory in rodents. The labyrinth is made of opaque gray polyvinyl chloride (PVC). Each arm is 40 cm long, 13 cm high and 3 cm wide at its base and 10 cm at its top. The arms converge towards one another, with an equal angle between the different arms. In brief, each mouse is placed at the end of an arm and left free to move for an 8-minute session. The mouse entries in each of the arms, including the arm at which it was dropped, are recorded. An alternation is defined as the successive entry of the animal into three different arms. Hence, the maximum number of alternations is the total number of entries in each of the arms minus 2 and the percentage of alternation is calculated according to the formula:

$$\% Alt = number of alternations completed / number {d}^{′} maximum alternations \times 100$$

The animal data showing extreme behaviors (percentage of alternation <20% or >90%) are not taken into account in the calculations. The attrition rate is usually 5%. Under normal conditions, a mouse will spontaneously alternate the explorations of each arm. A mouse with impaired memory and/or orientation capabilities will have its alternation percentage decreased.

### 1.4. Statistical Analyses-Calculation of the Combination Index (CI)

### Statistical Analyses

The analyses were performed using Prism v5.0 (GraphPad Software, San Diego, CA, USA). Data were analyzed using unidirectional variance analyses (ANOVA, F-value), followed by a Dunnett test or non-parametric Kruskal-Wallis ANOVA (H-value) followed by a Dunn comparison test. The statistical significance levels are p<0.05, p<0.01 and p<0.001.

### Combination Index

The nature of the interaction of two compounds at a given effect level was assessed by isobologram analysis (Martin et al., 2020; Mauritius, 2016). The representation of the isobolograms follows the Fraser concept (1872). The dose-response curves follow a biphasic effect, only the ascending part of the curve was taken into account in the calculations. The concentration required to produce a given effect (for example, where ICx=IC50) is determined for the compound A (ICx,A) and the compound B (ICx,B) and indicated on the x- and y-axes of a two-coordinate plot, forming the two points, (ICx,A, 0) and (0, ICx,B). The line connecting these two points is the additive line. In a mixture of drugs A+B, the concentrations of A and B contained in the combination that produce the same effect are represented by the coordinates (CA,x, CB,x). The combination index (CI) is calculated as follows:

$$IC = C A , x / ICx , A + C B , x / ICx , B$$

where CA,x and CB,x are the concentrations of the compounds A and B used in a combination that generates x % of the maximum combination effect.

CI is the combined index. ICx,A and ICx,B are the concentrations of the compounds A and B required alone to produce x % of the maximum effect. A CI less than/equal to/greater than 1 indicates synergy/additiveness/antagonism respectively. To calculate the CI based on the representation of the isobolograms, the percentages of alternation and passive avoidance latencies were expressed as percentage protection (PP) for each treatment group with PP (V/V) set to 100% and PP (Aβ₂₅₋₃₅/V) set to 0% (Martin et al., 2020; Mauritius, 2016).

### 2. Results

### 2.1. Synergistic Effect of a FENM-iChE Combination on the Short-Term Working Memory (YMT), in an AB Intoxication Model

Data confirm the anti-amnesic effects of FENM and DPZ on Aβ₂₅₋₃₅ oligomer-induced disorders on the short-term working memory.

Sub-effective doses, i.e. doses of compounds at which, when administered alone, an effect on FNEM-induced disorders at the limit of significance is observed or doses immediately below the first significantly active dose, were first investigated.

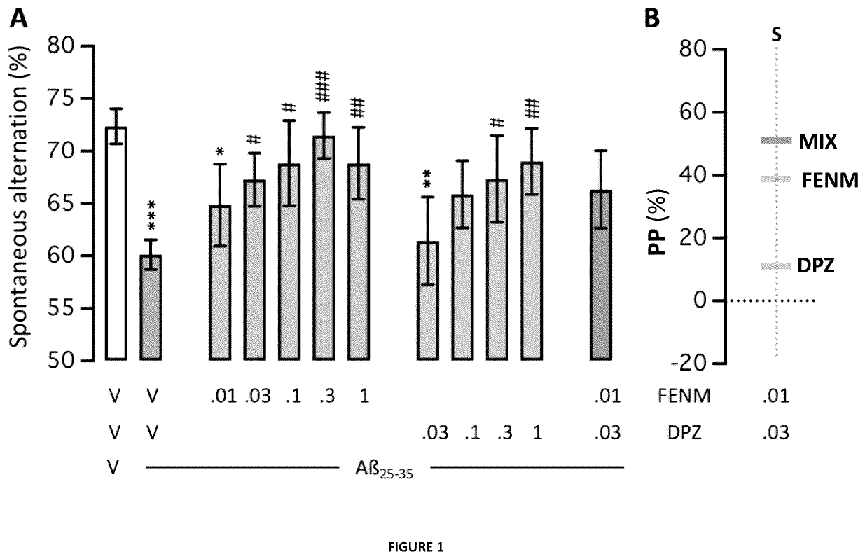

The performances of mice intoxicated with Aβ₂₅₋₃₅ and administered with FENM alone is significantly improved compared to the control as of 0.03 mg/Kg (FIG. 1). The improvement observed for the mice administered at a dose of 0.01 mg/Kg is at the limit of statistical significance. As of 0.03 mg/kg, FENM significantly improves the percentage of alternation of mice intoxicated with Aβ₂₅₋₃₅; furthermore, these performances are not significantly different from those of non-intoxicated mice. The administration of donepezil alone also allows for an improvement of the mnesic abilities of the animals measured by this test. However, no improvement was observed at 0.01 mg/Kg (not shown). Administered at 0.03 mg/kg, DPZ does not confer a statistically significant improvement on the performances of the intoxicated animals. Administered at 0.1 mg/kg, DPZ confers an improvement in mnesic symptoms, since the performances of the intoxicated and treated animals are not significantly different from those of the non-intoxicated and untreated animals; however, these performances are also not significantly different from those observed in untreated intoxicated mice.

The combined treatment of the intoxicated mice with FENM and DPZ at 0.01 and 0.03 mg/kg respectively results in an improvement in the percentage of alternation which is not significantly different from that of the non-intoxicated mice. The FENM/DPZ molar ratio in this combined treatment is 0.5.

The combination index data for these experiments are listed in Table 3 hereinbelow.

TABLE 3

| Treatment (mg/kg IP) | PP (%) | C_{x, FENM} | C_{x, DPZ} | CI |
|---|---|---|---|---|
| FENM (0) | 0.0 ± 18.2 | | | |
| FENM (0.01) | 38.5 ± 31.9 | | | |
| FENM (0.03) | 58.4 ± 20.8 | | | |
| FENM (0.1) | 71.2 ± 33.3^{†} | | | |
| DPZ (0) | 0.0 ± 18.2 | | | |
| DPZ (0.03) | 10.6 ± 34.0 | | | |
| DPZ (0.1) | 46.9 ± 26.4 | | | |
| DPZ (0.3) | 58.9 ± 33.7^{•} | | | |
| FENM (0.01) + DPZ (0.03) | 50.7 ± 30.2 | 0.063 ± 0.016 | 0.236 ± 0.066 | 0.30 ± 0.08 |

PP: percentage protection; CI: combination index.
C_{x, FENM} and C_{x, DPZ} were calculated from the regression line of the dose-response curves of each of the compounds alone:
^{†}y = 841.34x, R² = 0.7817;
^{•}y = 224.69x, R² = 0.8819.

An IC of 0.3 (Table 3) is measured for the FENM-DPZ combination at sub-effective concentrations. This IC reflects a strong synergistic interaction between the FENM and the DPZ, as illustrated by the percentage scale of protection materialized in FIG. 1. This strong synergistic interaction results in a percentage of protection that is not reached for mice administered with the molecules alone, regardless of the doses. In the spontaneous alternation test, it translates into an improvement in the mnesic performances of the intoxicated animals (FIG. 1) treated with the FENM-DPZ combination, in comparison with the animals treated with the molecules alone.

### 2.2. Synergistic Effect of a FENM-iChE Combination on the Long-Term Memory (PAT), in an Aβ Intoxication Model

The data confirm the anti-amnesic effects of FENM and DPZ on Aβ₂₅₋₃₅ oligomer-induced disorders on the long-term working memory.

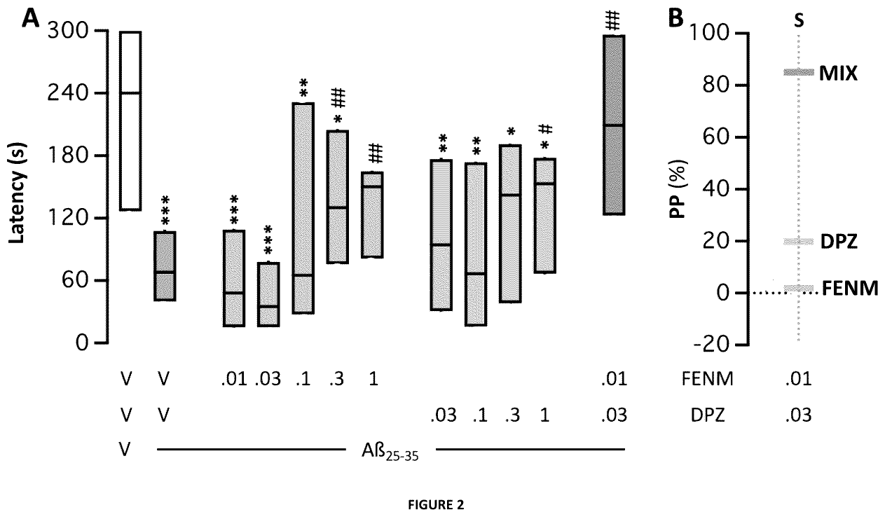

The mice intoxicated with Aβ₂₅₋₃₅ and administered with the doses of 0.3 mg/kg or 1 mg/kg of FENM IP show performances that are not significantly different from the control, non-intoxicated, untreated mice (FIG. 2); these performances are significantly different from the untreated, intoxicated mice. Administration of FENM at lower doses does not protect mice from mnesic disorders due to Aβ₂₅₋₃₅ intoxication. Only the highest dose of DPZ tested (1 mg/kg) proves to be effective in counteracting the amnesic effects of intoxication with Aβ₂₅₋₃₅ oligomers: the latency time of animals is significantly different from that of untreated intoxicated animals. Nevertheless, the improvement is not sufficient to fully restore the performances of the animals since the latency time for these remains significantly different from the latency time of untreated non-intoxicated animals.

The combined administration of FENM and DPZ at the lowest and most ineffective doses of FENM and DPZ, 0.01 and 0.03 mg/kg respectively, results in a significant protection of the long-term memory capacities of the intoxicated mice compared to those of the untreated intoxicated mice (FIG. 2). In a particularly interesting manner, these doses are more than one order of magnitude lower than the first effective doses identified in this test, reflecting a particularly important synergistic effect of the compounds on the long-term memory.

As indicated in Table 4 hereinbelow, such a protection at low doses of compounds, although substantially low, could be explained by a particularly significant synergy of each of the compounds, supported by the particularly low combination index, which reflects a particularly significant synergistic effect.

TABLE 4

| treatment (mg/kg IP) | PP (%) | C_{x, FENM} | C_{x, DPZ} | CI |
|---|---|---|---|---|
| FENM (0) | 0.0 ± 7.2 | | | |
| FENM (0.01) | 1.4 ± 18.3 | | | |
| FENM (0.03) | −12.2 ± 16.8^{††} | | | |
| FENM (0.1) | 27.8 ± 24.8 | | | |
| FENM (0.3) | 52.9 ± 14.2 ^{†} | | | |
| DPZ (0) | 0.0 ± 7.2 | | | |
| DPZ (0.03) | 19.7 ± 17.2 | | | |
| DPZ (0.1) | 19.6 ± 21.6 | | | |
| DPZ (0.3) | 42.2 ± 21.4 | | | |
| FENM (0.01) + DPZ (0.03) | 84.9 ± 22.8 | 0.469 ± 0.076 | 0.563 ± 0.095 | 0.07 ± 0.01 |

PP: percentage protection; CI: combination index.
C_{x, FENM} and C_{x, DPZ} were calculated from the regression line of the dose-response curves of each of the compounds alone:
^{†} y = 180.98x, R² = 0.891;
^{•}y = 150.83x, R² = 0.898.
^{††}negative PP results in negative C_{x, FENM}.

### 3. Conclusion

These data show a synergy of the protective effects of FENM combined with those of an iChE, for both the short-term working memory and the long-term working memory. Furthermore, this synergy is specific to the use of FENM since it is not observed for combinations in which memantine replaces FENM.

These results allow considering the use of FENM-iChE combinations in patients currently without an approved therapeutic solution for the treatment of the mnesic symptoms of AD or related pathologies, such as patients in the early-stage of the disease. Also, the observed synergy which allows obtaining a therapeutic effect at particularly low doses of the compounds for which little or no adverse effect is expected.

### REFERENCES

Chumakov I, Nabirotchkin S, Cholet N, Milet A, Boucard A, et al., Combining two repurposed drugs as a promising approach for Alzheimer's disease therapy. Sci Rep. 2015 Jan. 8; 5:7608.
Couly S, Denus M, Bouchet M, Rubinstenn G, Maurice T. Anti-Amnesic and Neuroprotective Effects of Fluoroethylnormemantine in a Pharmacological Mouse Model of Alzheimer's Disease. Int J Neuropsychopharmacol. 2021; 24:142-157.
Fraser T R. Lecture on the antagonism between the actions of active substances. Br Med J. 1872; 2:485-7.
Kilkenny C, Browne W, Cuthill I C, Emerson M, Altman D G; NC3Rs Reporting Guidelines Working Group. Animal research: reporting in vivo experiments: the ARRIVE guidelines. Br J Pharmacol. 2010; 160:1577-9.
Maekawa Y, Hasegawa S, Ishizuka T, Shiosakai K, Ishizuka H. Pharmacokinetics and Bioequivalence of Memantine Tablet and a New Dry Syrup Formulation in Healthy Japanese Males: Two Single-Dose Crossover Studies. Adv Ther. 2019 October; 36 (10): 2930-2940.
Martin P, de Witte PAM, Maurice T, Gammaitoni A, Farfel G, Galer B. Fenfluramine acts as a positive modulator of sigma-1 receptors. Epilepsy Behav. 2020; 105:106989.
Maurice T, Hiramatsu M, Itoh J, Kameyama T, Hasegawa T, Nabeshima T. Behavioral evidence for a modulating role of sigma ligands in memory processes. I. Attenuation of dizocilpine (MK-801)-induced amnesia. Brain Res. 1994a; 647:44-56
Maurice T, Lockhart B P, Privat A (1996) Amnesia induced in mice by centrally administered β-amyloid peptides involves cholinergic dysfunction. Brain Res. 706:181-93.
Maurice T, Su T P, Parish D W, Nabeshima T, Privat A. PRE-084, a sigma selective PCP derivative, attenuates MK-801-induced impairment of learning in mice. Pharmacol Biochem Behav. 1994b; 49:859-69.
Maurice T. Protection by sigma-1 receptor agonists is synergic with donepezil, but not with memantine, in a mouse model of amyloid-induced memory impairments. Behav Brain Res. 2016; 296:270-278.
Meunier J, Ieni J, Maurice T. The anti-amnesic and neuroprotective effects of donepezil against amyloid beta25-35 peptide-induced toxicity in mice involve an interaction with the sigma1 receptor. Br J Pharmacol. 2006; 149:998-1012.
Remington: The Science and Practice of Pharmacy (23rd ed.), Ed. A Adeboye Adejare, 2020.
Rogers S L, Friedhoff L T. Pharmacokinetic and pharmacodynamic profile of donepezil HCl following single oral doses. Br J Clin Pharmacol. 1998 November; 46 Suppl 1 (Suppl 1): 1-6.
Rossetti H C, Munro Cullum C, Hynan L S, Lacritz L H. The CERAD Neuropsychologic Battery Total Score and the progression of Alzheimer disease. Alzheimer Dis Assoc Disord. 2010; 24 (2): 138-142.
Stahl P H, Wermuth C G. Pharmaceutical Salts: Properties, Selection, and Use. Wiley, 2011. 388 pages.
Zhang, N., Zheng, X., Liu, H. et al. Testing whether the progression of Alzheimer's disease changes with the year of publication, additional design, and geographical area: a modeling analysis of literature aggregate data. Alz Res Therapy 12, 64 (2020).

1. A composition comprising a synergistic combination of 3-(2-fluoroethyl) adamantan-1-amine (FENM) or any one of its pharmaceutically-acceptable salts and at least one acetylcholinesterase inhibitor or any one of its pharmaceutically-acceptable salts.

2. The composition according to claim 1, wherein said at least one acetylcholine esterase inhibitor is selected from among donepezil, galantamine, rivastigmine, tacrine, or any one of their pharmaceutically-acceptable salts.

3. The composition according to claim 1, wherein the FENM dose is compatible with a FENM dose less than or equal to 20 mg/day.

4. The composition according to claim 1, wherein said at least one acetylcholinesterase inhibitor is donepezil which is present at a dose compatible with a dosage less than or equal to 10 mg/day.

5. The composition according to claim 1, wherein said at least one acetylcholinesterase inhibitor is the rivastigmine which is present at a dose compatible with a dosage less than or equal to 3 mg/day.

6. The composition according to claim 1, wherein said at least one acetylcholinesterase inhibitor is galantamine which is present at a dose compatible with a dosage less than or equal to 16 mg/day.

7. The composition according to claim 1, wherein the FENM/at least one acetylcholine esterase inhibitor molar ratio is less than or equal to 4.

8. A pharmaceutical composition comprising the composition according to claim 1 and one or more suitable excipients.

9. A method of treating a pathology selected from among tauopathies, synucleinopathies, amyloidopathies, Alzheimer's disease, Parkinson's disease, multiple system atrophy, Huntington's disease, posterior cortical atrophy, Pick's disease, epilepsy, vascular dementia, frontotemporal dementia, dementia with Lewy bodies, amyotrophic lateral sclerosis comprising administering, to a subject in need thereof, an effective amount of the composition according to claim 1.

10. A method of treating a pathology selected from among tauopathies, synucleinopathies, amyloidopathies, Alzheimer's disease, Parkinson's disease, multiple system atrophy, Huntington disease, posterior cortical atrophy, Pick's disease, epilepsy, vascular dementia, frontotemporal dementia, dementia with Lewy bodies, amyotrophic lateral sclerosis, comprising:
administering to a subject in need thereof, an effective amount of 3-(2-fluoroethyl) adamantan-1-amine (FENM), or a pharmaceutically-acceptable salt thereof, in synergistic combination with at least one acetylcholinesterase inhibitor selected from among donepezil, galantamine, rivastigmine, tacrine, or any one of their pharmaceutically-acceptable salts.

11. The composition according to claim 7, wherein the FENM/at least one acetylcholine esterase inhibitor molar ratio is less than or equal to 3.

12. The composition according to claim 11, wherein the FENM/at least one acetylcholine esterase inhibitor molar ratio is less than or equal to 2.

13. The composition according to claim 12, wherein the FENM/at least one acetylcholine esterase inhibitor molar ratio is less than or equal to 1.


SEQUENCE LISTING (1 sequence)

SEQ ID NO: 1 - protein, 11 aa, synthetic construct
GSNKGAIIGLM